(19) United States
(12) Patent Application Publication
(10) Pub. No.: US 2009/0145442 A1
(43) Pub. Date: Jun. 11, 2009

(54) Cranio-Cervical Extension Pillow with Dual Arcs

(75) Inventors: Lawrence E. Hecox, Ventura, CA (US); David Eckhous, Long Beach, CA (US); Blain Tomlinson, Long Beach, CA (US)

Correspondence Address: FISH & ASSOCIATES, PC;ROBERT D. FISH, 2603 Main Street, Suite 1000, Irvine, CA, 92614-6232, US

(73) Assignee: CPAIR, INC., San Clemente, CA (US)

(21) Appl. No.: 12/327,363

(22) Filed: Dec. 3, 2008

Related U.S. Application Data
(60) Provisional application No. 60/992,842, filed on Dec. 6, 2007.

(51) Int. Cl. A61F 5/37

(52) U.S. Cl. 128/845

(57) ABSTRACT

A device comprising a supporting material shaped with dual arcs is presented. When the device is placed beneath a person's neck, the arcs cradle the neck and the device provides proper capital extension for CPR. One of the arcs has a smaller curvature to cradle adult necks while the other arc has a larger curvature to cradle smaller neck of children. In a preferred embodiment, the device comprises a pillow having a saddle shape that can used to support a victim's neck during CPR. In some embodiments, the pillow includes electronics that can guide a CPR provider through CPR procedures.

This application claims the benefit of priority to U.S. provisional application 60/992,842 filed Dec. 6, 2007. This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The field of the invention is cranio-cervical support technology.

### BACKGROUND

Cardiopulmonary resuscitation (CPR) procedures require tilting a victim's head back to open the trachea to improve air flow to the lungs. Ideally, a CPR provider should be trained and knowledgeable on the proper method of providing capital extension. However, in most cases where the victim requires immediate CPR, the provider often forgets their training, or simply doesn't have sufficient training to provide proper capital extension. The result can render the CPR procedure ineffective.

Consequently, it is very helpful for a CPR provider to have access to a device that maintains a proper capital extension of a victim during CPR. A preferred device should also instruct the provider how to use the device, including instructions on positioning the victim's neck, cradling the victim's neck, keeping the victim's shoulders in contact with the ground, and providing proper capital extension to open the trachea. Ideally, such a device would also accommodate a broad spectrum of people from small children having narrow necks to large adults having thick necks.

Others have put forth effort to aid the breathing of individuals under various circumstances. For example, international patent application WO 2006/043704 to Sharp et al. and U.S. Patent Application Publication 2008/0173312 to Peake et al. provide for devices including pillows to open a person's air way that suffers from sleep apnea. Another example includes U.S. Pat. No. 6,644,228 to Pi, where Pi describes a pillow for use in opening a person's air way for intubation. U.S. Patent Application Publication 2003/0062040 to Lurie et al. makes further progress by providing a headrest that can include a communication device to send signal to an emergency service. However, these and other known references fail to address all the needs for the features outlined above.

Thus, there is still a need for a device that correctly provides capital extension for a person's neck to open their trachea for CPR.

### SUMMARY OF THE INVENTION

The present invention provides apparatus, systems and methods in which a victim requiring CPR can be properly positioned so their trachea is receptive to air flow.

One aspect of the inventive subject matter includes cranio-cervical extension pillows that offer CPR providers a way to properly provide capital extension to a victim's neck during CPR.

In a preferred embodiment, the pillow has a support material shaped to cradle the victim's neck. Most preferably, the cradle has a first arc having a small curvature (relatively large radius) to accommodate larger necks, and a second arc having a larger curvature (relatively smaller radius) to accommodate victims having smaller necks. The first and second arcs form a cradle that receives a victim's neck. The pillow is preferably sized and dimensioned to allow the victim's head or shoulders to remain in contact with a surface while CPR is preformed. Preferred pillows include electronics housed within the pillow that include hardware or software configured to provide CPR instructions to the CPR provider.

Various objects, features, aspects and advantages of the inventive subject matter will become more apparent from the following detailed description of preferred embodiments, along with the accompanying drawings in which like numerals represent like components.

### BRIEF DESCRIPTION OF THE DRAWING

FIG. 1A is a schematic of a front, isometric view of a cranio-cervical extension pillow having dual arcs.

FIG. 1B is a schematic of a back, isometric view of a cranio-cervical extension pillow having dual arcs.

FIG. 1C is a schematic of a top view of a cranio-cervical extension pillow having dual arcs.

FIG. 1D is a schematic of a front view of a cranio-cervical extension pillow having dual arcs.

FIG. 2 is a schematic of an exploded view of a cranio-cervical extension pillow.

### DETAILED DESCRIPTION

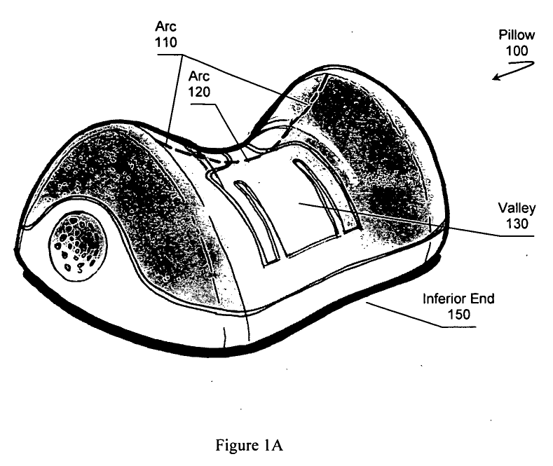

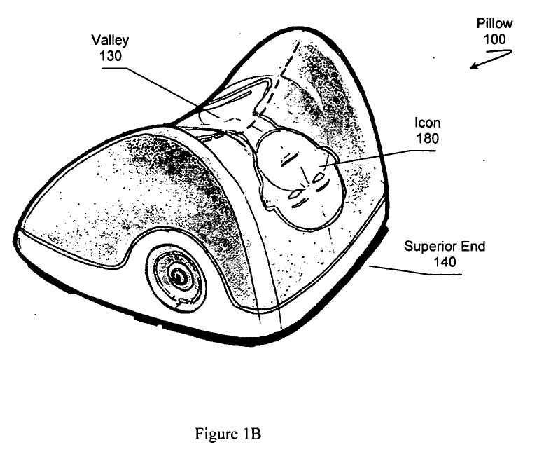

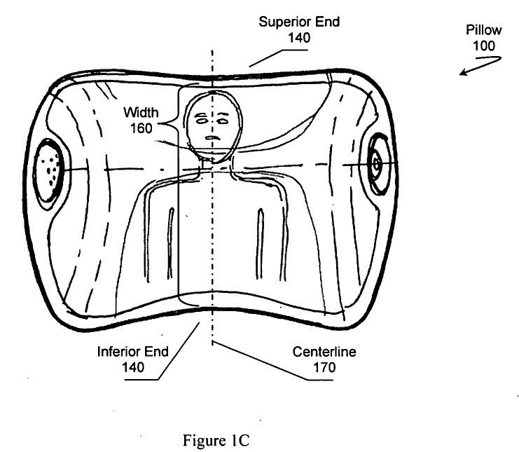

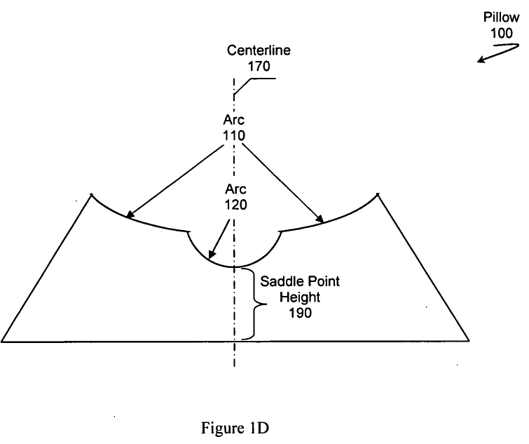

In FIGS. 1A, 1B, 1C, and 1D, a cranio-cervical extension pillow comprises supporting material having a saddle shape.

FIGS. 1A and 1B are schematics of front and back isometric views, respectively, of a possible embodiment of pillow with dual arcs. The saddle shape of a preferred pillow is roughly defined by arc 110 and arc 120 running the length of the pillow laterally from left to right and by a valley 130 running from a superior end 140 to an inferior end 150 of the pillow. The saddle shape cradles a victim's neck while maintaining proper capital extension during CPR.

In a preferred embodiment, the superior end 140 of the valley 130 allows the crown of a victims head to touch a surface on which the victim is lying. Additionally, the inferior end 150 of valley 130 allows the victim's shoulders to remain in contact with the surface. Proper contact of the head and shoulders is desired to ensure CPR compression strokes have maximal efficiency. If the victim's head and shoulders do not have proper contact with the surface, the victim's body could be cushioned by the pillow during compression strokes, which could unnecessarily rob the stroke of its energy or its effectiveness.

FIG. 1C is a schematic of a top view of a cranio-cervical extension pillow. Valley 130 of the pillow running from superior end 140 to inferior end 150 of the pillow is preferably anatomically countered to fit the lordosis of an average 70 kg man.

To accommodate an even larger portion of the population, a width 6 of the pillow as shown in FIG. 1C, and as measured along a centerline 170 of valley 130, preferably varies from 8-16 cm. In the preferred embodiment, width 6 is approximately 10-14 cm. Unless the context dictates otherwise, all ranges herein are to be interpreted as being inclusive of their endpoints.

Preferred embodiments include markings on the pillow to indicate proper positioning of the pillow beneath a victim's neck. In the preferred embodiment, the markings include an icon comprising a raised rib showing how a person should be positioned on the pillow. In other embodiments, the pillow can include words indicating which side of the pillow is placed toward the foot or the head of the victim. In FIGS. 1B and 1C, icon 180 shows a person's body and head properly positioned on the pillow.

FIG. 1D is a schematic of a front view of a cranio-cervical extension pillowing having dual arcs. The arcs are exaggerated in FIG. 1D to illustrate differences in the respective curvatures. As used herein, “arc” means any concave or convex curve. Although an arc can include a mathematically defined curve, in the preferred embodiment the arcs are anatomically contoured to accommodate large portions of the population. Additionally, as used herein “curvature” means qualitatively or quantitatively how much an arc curves and is not limited only to a mathematical definition of curvature.

Preferably, arc 110 and arc 120 are roughly co-planar, and run laterally along the crest of the pillow. Arc 110 has a small curvature (a larger radius) to accommodate larger victims, preferably having a weight greater than 45 kg. Arc 120 has a larger curvature (smaller radius) to accommodate smaller victims, preferably having a weight from 18 to 45 kg. The approximate centers of curvature of the arcs lie roughly along a pillow centerline 170 running from superior end 140 to inferior end 150 and bisecting the pillow left to right. One should appreciate that the use of mathematical language to describe the pillow is presented strictly for clarity, and should not be interpreted as limiting the shape, curvature, or other attribute of contemplated pillows.

FIG. 1D also depicts a saddle point height 190, measured from the surface on which the pillow rests to the closest point on arc 120. In a preferred embodiment, saddle point height 190 is in the range 2-10 cm. In a more preferably embodiment, saddle point height 190 is approximately in the range 4-8 cm. Having a proper saddle point height ensures a victim's cervical spine has proper capital extension or otherwise support optimal air flow.

Those of ordinary skill in the art should appreciate a pillow with dual arcs and a saddle shape can accommodate a large portion of the population. Preferably the pillow is shaped to accommodate at least 60% of the general population and more preferably at least 80% of the general population.

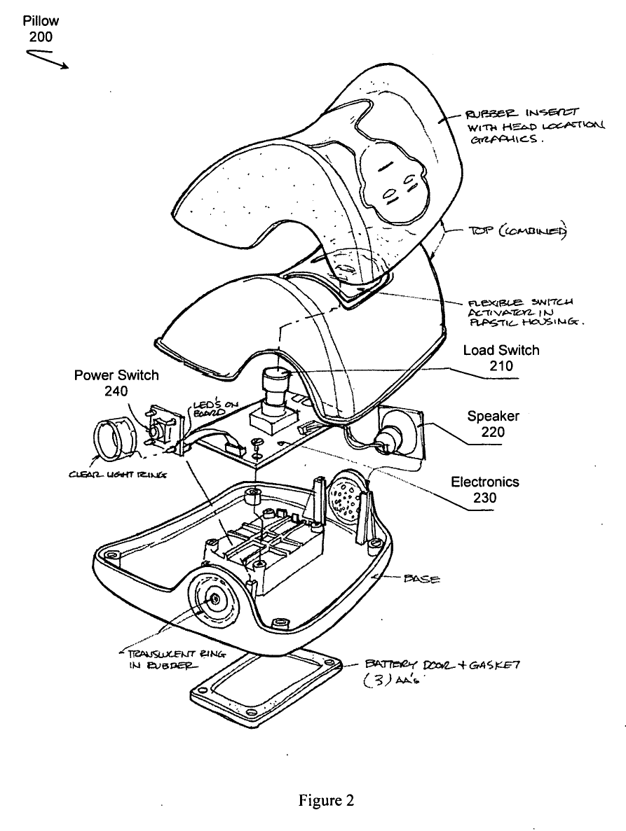

In FIG. 2, an exploded view of a possible embodiment of a cranio-cervical extension pillow 200 that includes electronics 230 within the pillow.

Pillow 200 is constructed of one or more supporting materials that form a structure for supporting the weight of a victim's neck. Preferred supporting material includes suitably rigid plastic. Preferably the surface of pillow 200 is coated with a soft rubber form placed over the rigid structure of the rigid structure of pillow 200. One should note that one or more other supporting material can also be used without departing from the scope of the inventive subject matter. For example, pillow 200 could be constructed from wood, metal, hard rubbers, or any combination of materials.

The plastic case in a preferred embodiment defines cavity that can be used to package the electronics 230 preferably activated by power switch 240. In the preferred embodiment the electronics comprises hardware or software configured to aid a CPR provider in performing CPR. Preferred electronics 230 comprise microprocessor and a memory storing software instructions to be executed by the microprocessor. In a preferred embodiment, the software instructions configure electronics 230 to provide audio instructions to CPR provider via speaker 220. In such embodiments, the memory can store audio data as well as software instructions.

It is also contemplated that pillow 200 can also include one or more sensors. For example, in some embodiment pillow 200 includes load switch 210 to detect when a victim is properly placed on pillow 200, switch 210 becomes depressed, which signals the microprocessor to being executing appropriate software instructions. Switch 210 can also provide a signal to the microprocessor upon detecting a compression stroke of the provider. Other contemplated sensors include pulse sensor, respiratory sensor, GPS sensor, or other sensors that could aid the CPR provider.

Some embodiments of pillow 200 also include additional communication capabilities. It is contemplated that pillow 200 could be equipped with cell phone transceivers (e.g., for CDMA, GSM, etc.) to allow automatic calls to 911 or other emergency personal. Additionally, pillow 200 could be equipped with wireless network capabilities (e.g., 802.11, 802.16, etc.) to provide connectivity to data networks. Network connectivity can provide for supplying remote emergency personal with biometric data of the victim, or possibly voice communication.

The disclosed inventive subject matter represents foundational elements for creating product that can be of use in emergency situations. It is contemplated that the disclosed devices could be deployed in high traffic, public locations (e.g., airports, bus terminals, schools, malls, etc.). One should note that the concepts of selling, customizing, configuring, or deploying such devices also falls within the scope of the inventive subject matter.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A cranio-cervical extension pillow comprising:
a support material having a first concave arc, and a second concave arc different from the first arc and approximately parallel to the first arc; and
wherein the first and the second arc form a cradle that receives a person's neck; and
wherein the pillow provides capital extension when placed beneath the person's neck.

2. The pillow of claim 1, wherein the second arc has a larger curvature than the first arc.

3. The pillow of claim 2, wherein the first arc has a longer length than the second arc.

4. The pillow of claim 1, wherein the support material further has a valley, which cooperates with the arc to form a saddle shape.

5. The pillow of claim 4, wherein the valley runs from a superior end of the pillow to an inferior end of the pillow.

6. The pillow of claim 1, wherein a width of the pillow, taken along a centerline running from a superior end of the pillow to an inferior end of the pillow, is between 8 cm and 16 cm.

7. The pillow of claim 6, wherein the width is between 10 cm and 14 cm.

8. The pillow of claim 1, wherein a maximum height of the pillow at an inferior to superior centerline is between 2 cm and 10 cm.

9. The pillow of claim 8, wherein the height is between 4 cm and 8 cm.

10. The pillow of claim 1, wherein the support material defines a housing having a cavity.

11. The pillow of claim 10, further comprising electronics disposed within the cavity.

12. The pillow of claim 11, wherein the electronics include a speaker.

13. The pillow of claim 11, wherein the electronics includes a memory.

14. The pillow of claim 11, wherein the electronics includes a sensor.

15. The pillow of claim 14, wherein the sensor comprises a compression sensor.

16. The pillow of claim 11, further comprising a wireless transceiver.

17. The pillow of claim 1, further comprising a marking on the support material that indicates a proper orientation of the pillow with respect to a person.
